(19) United States
(12) Patent Application Publication
(10) Pub. No.: US 2020/0390986 A1
(43) Pub. Date: Dec. 17, 2020

(54) INDUCTION POWERED VAPORIZER DEVICE CAPABLE OF UTILIZING SOLID OR LIQUID PRODUCTS

(71) Applicant: Jamie Rosen, Las Vegas, NV (US)

(72) Inventor: JAMIE ROSEN, Las Vegas, NV (US)

(73) Assignee: DR. DABBER, INC., Las Vegas, NV (US)

(21) Appl. No.: 16/437,613

(22) Filed: Jun. 11, 2019

(51) Int. Cl. A61M 11/04; H05B 6/06; H05B 6/10

(52) U.S. Cl. CPC A61M 11/041; H05B 6/06; H05B 6/10; A61M 2205/587; A61M 2205/3331; A61M 2205/36; A61M 2205/8206; A61M 2205/0211

(57) ABSTRACT

Present disclosure relates generally to an improved design for an induction powered vaporizer that is capable of vaporizing both liquid and solid products, specifically liquid as in oil-based products, and solid as in leaf-based products. The device and method thereof incorporate unique and novel induction heating, the air-flow design, and in the circuitry control of the induction vaporizer. Other improvements in the design of the induction powered vaporizer included herein.

### CROSS-REFERENCE TO RELATED APPLICATIONS

29/643,605, Filing Date: Apr. 10, 2018, Art Unit 2913, Confirmation No. 2846

### FEDERALLY SPONSORED RESEARCH

None

### SEQUENCE LISTING

None

### TECHNICAL FIELD OF INVENTION (FOI)

This present disclosure relates generally to an improved design for an induction powered vaporizer device and method thereof, that is capable of vaporizing both oil-based products, and leaf-based products. More specifically, this invention relates to a unique and novel implementation that utilizes induction heating, the air-flow design, and in the circuitry control of the induction vaporizer. Other improvements in the design and implementation of the induction powered vaporizer device are also included in this disclosure.

### BACKGROUND

Various types of vaporizing devices are commonly known. Some devices are designed to enable an individual to inhale and absorb recreational compounds without the harmful effects associated with inhalation via conduction. Additionally, vaporizers can be used to disperse pleasant aromas or even applied in medical contexts.

Generally, vaporizing units are composed of a mouthpiece, a storage chamber, and a heating element. Storage chambers of the vaporizing material come in multiple configurations such as those that require refilling after each use and those that contain a larger chamber capable of storing larger amounts of oil or other vaporizing material. Additionally, storage chambers are configured to receive one type of vaporizing material, usually oil or leaf-based material. Heating elements are divided into two general categories, those that require conduction to heat and those that heat via convection.

Multiple design and methods to improve desirable qualities such as increased volume of exhaled vapor cloud, reduced battery usage, reduced vaporizing material consumption, reduction in size, and prevention of leaking have been attempted through various prior art. However, each prior art failed to overcome specific challenges to create a handheld vaporizing apparatus with a unique airflow path to maximize exhaled vapor cloud based on a reduction in needed materials and energy with a sufficiently simple design to enable ease of production.

For example, Magnon U.S. Pat. No. 8,291,918 discloses a portable vaporizing unit comprised of a hollow tube with a user control interface capable of controlling the temperature for either conduction or convection heating of a fluid. This invention ignores the potential to use a predefined airflow path to manage temperature an essential variable in creating a pleasant vaporizing experience. This invention can only use fluid as a vaporizing material. Additionally, the disclosed design is needlessly complex and leads to increased production, assembly, and repair costs. In opposition, the present invention minimizes extemporaneous parts and is a simplified design permitting lower unit cost production and reduces the likelihood of malfunction by reducing the number of parts capable of malfunctioning while increasing functionality through the addition of leaf-based vaporizing materials and defined air flow path, permitting vapor and ambient air to be effectively controlled.

Other prior art discloses similarly deficient designs. For example, Rosenthal U.S. Pat. No. 7,997,280 discloses a pen shaped vaporizing device including an elongated airflow pathway with a plurality of ambient air intake apertures and concurrent heating element. This prior art fails to effectively separate ambient air from vapor, effectively resulting in a relative increased temperature resulting in degradation including decreasing the concentration, quality, and enjoyment of the vaporized material. Additionally, the broad surface of the heating element consumes a great deal of vaporizing material with each use, degrading the efficiency ratio of vaporizing material to vapor produced. The present invention overcomes such shortcomings via a defined airflow path with a smaller heating element permitting increased vaporization without material waste.

It is an object of the present invention to provide a portable electronic induction vaporization device, that utilizes proprietary circuitry to provide induction heating to an inserted induction cup, which directly heats the desired leaf or oil material.

It is still another object of this invention to provide an induction cup housed in a glass top creating a unique vaporization chamber to protect electronics of device from damaging vapor byproducts.

It is still another object of this invention to provide a system which vaporizes either liquid or solid product by induction heating of induction cups coated or bonded with ceramic, and yet another embodiment wherein the glass induction cup has a bonded metal ring to enable the inert material to maintain taste and purity of vapor.

It is still a further object of this invention to provide a system that allows for the glass top and induction circuitry to be housed in a plastic shell supporting the chassis and cover plates.

It is another object of the invention to provide a system which contains a replaceable battery pack, user interactive LED display, and multiple user activated buttons or switches.

It can be seen, therefore, that there is a device needed to enable a portable method of vaporizing both oil-based products and leaf-based products in one apparatus for induction vaporization protected with a simple, cost-effective industrial design.

### SUMMARY

This present disclosure has overcome the disadvantages and shortcomings of the prior art by implementing an induction powered vaporizer with an induction chamber with inert material and no exposed electronics that is capable of vaporizing both oil-based products, and leaf-based products. This present invention provides a portable electronic induction vaporization device that utilizes proprietary circuitry to provide induction heating to an inserted cup, which directly heats the desired leaf or oil material of the user. The present disclosure and embodiments utilize a method of induction heating, the air-flow design, and in the circuitry control of the induction vaporizer.

This present disclosure includes: an induction heating system, induction cups, anaerobic vaporization, and proprietary induction circuitry with predictive temperature measurement. The heating system allows an induction cup to be heated without any electrical connections directed attached to the induction cup.

The present disclosure also has several other features that are not essential to functionality but are integrated into the apparatus as design elements including: an attachable percolator, removable battery pack, plug and play vaporizer capability, and a programmable maintenance port.

The present invention is a portable electronic induction vaporization device, that utilizes proprietary circuitry to provide induction heating to an inserted induction cup, which directly heats the desired leaf or oil material.

An induction cup is housed in a glass top element, which has a cavity referred to as a vaporization chamber that creates a unique vaporization chamber to protect the electronics of the device from vapor byproducts. This novel design results in zero exposed electronics to a defined vaporization chamber. This vaporization chamber is double-sealed to prevent any contamination of the vapor, and to protect the electronics from vapor and vapor by-products. Material is vaporized by the induction heating of induction cups, which are partially composed of metallic materials that are heated by induction. The induction cups are designed to enable the vaporized oil or leaf to only come in contact with inert materials These induction cups are coated or mostly composed of inert material to help maintain the taste and purity of the produced vapor regardless of the product as liquid or solid-leaf.

The induction cup and glass housing create a well, that results in a low-oxygen environment when heating occurs for both solid products or liquid products loaded into the induction cup. Additionally, the loaded liquid or solid products can either be preloaded into the induction cups, or can be loaded after an induction cups are placed in the device and that was pre-heated externally.

The induction cups are made from a metal, and can be coated or bonded with a ceramic, or in the alternative a non-metallic cup is bonded to a metal ring, where the non-metallic cup is the surface directly heating the loaded material.

Another embodiment of the present invention contains a non-metallic cup inserted into a metal induction cup, used as the vaporization cup, where the non-metallic cup is the surface directly heating the loaded material, but the non-metallic cup is itself being heated by the metal induction cup directly.

The present disclosure includes a glass top, along with an induction circuitry, housed in a plastic shell, which includes supporting chassis and cover plates. This shell also houses the replaceable battery pack which connects to the induction circuitry and powers the entire vaporizer device as described. The present device and method interacts with a user by way of a front facing LED display, with said LED display housed in a glass top, and is activated using a combination of a rocker power switch, along with three buttons profiled as a “Plus (+), Minus (−),” and a “Go” designation. The present device, in an embodiment variant, allows a battery pack to be externally charged with an appropriate chord and power interface.

A user will be able to adjust the settings of the present invention to control the heating settings and power induced into the induction cups. Specifically, that the heating profile, which is the temperature verses time curve, controlled by the device, and customized for the vaporization of either loose-leaf or oil-based materials. Further, a user can control the heating profile, which is the temperature verses time curve, customized for the vaporization of the type of induction cup being metal or non-metal.

The present invention to include a replaceable battery pack, user interactive LED display, and multiple user activated buttons or switches. Additionally, the device can operate without a replaceable battery pack, but being powdered directly from a wall-outlet or alternative power source.

The present invention wherein vapor can be directed through a water bubbler for filtration and cooling. The inner workings of the water bubbler can vary greatly by design, but achieve the same practical application of filtration and cooling. Further, the airflow to the induction cup can be restricted using an air limiting “carb” tool, which “carb” tool and the water bubbler are optional for use of the device, specifically the present invention can still produce vapor without these two elements.

These and other features, aspects, and advantages of the invention will be further understood and appreciated by those skilled in the art by reference to the following written specification, claims and disclosure drawings.

The present invention is a device which enables a portable method of vaporizing both oil-based products and leaf-based products in one apparatus for induction vaporization, which is protected with a simple, cost-effective industrial design.

### BRIEF DESCRIPTION OF DRAWINGS

An embodiment of the present invention will hereinafter be described in conjunction with appended drawings, provided to illustrate and not to limit the invention. Drawings included in this Provisional Patent Application are illustrative of the present disclosure, method, and designs but said drawings will be amended in the anticipated Non-Provisional Application. The Figure descriptions below rate the drawing intent and objective but do not relate specifically to any current drawing or embodiment of the present invention and the inventor reserves the right to update, modify, amend or revoke any of the depicted drawings in anticipation of a Non-Provisional Patent Application.

FIG. 001 (FIG. 1) depicts a combined frontal and side view of the present device with a glass percolator and dab tool accessory.

FIG. 002 (FIG. 2) depicts a frontal view of the present device without a glass percolator.

FIG. 003 (FIG. 3) depicts a cross-section and cut-out view of the present device.

FIG. 004 (FIG. 4) depicts an exploded view of the present device.

FIG. 005 (FIG. 5) depicts a cross-section view of the present invention with an induction assembly.

FIG. 006 (FIG. 6) depicts a cross-section view of the ceramic induction cup.

FIG. 007 (FIG. 7) depicts a cross-section view of the crystal induction cup.

FIG. 008 (FIG. 8) depicts a cross-section view of the present device displaying the seals of the vaporization chamber.

FIG. 009 (FIG. 9) depicts a cross-section of the anaerobic vaporization of materials inside the apparatus.

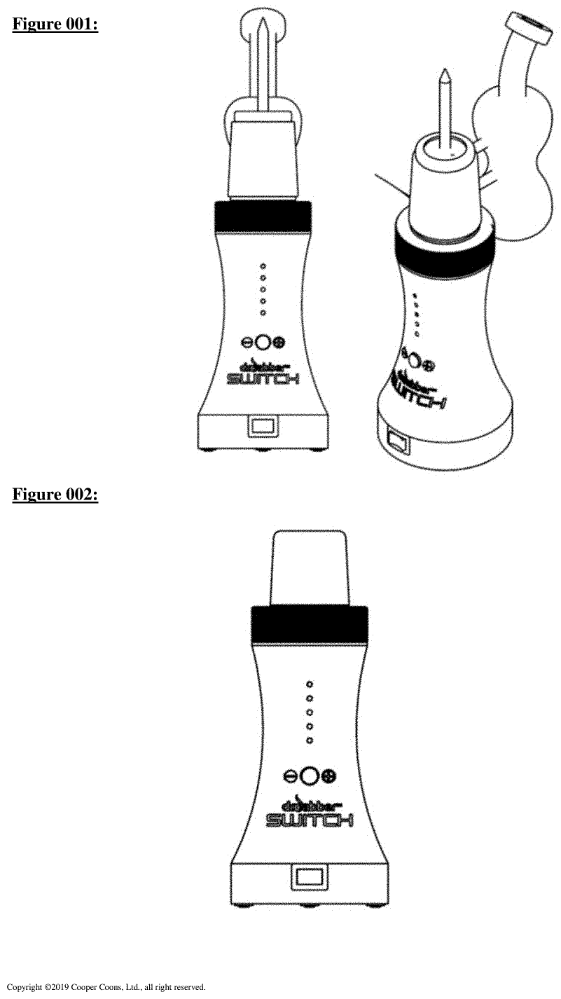

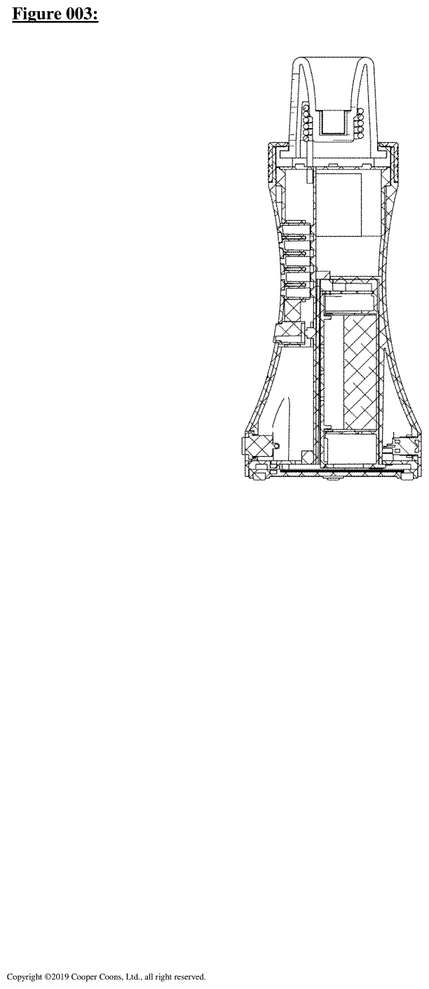

### DETAILED DESCRIPTION OF DRAWINGS

This present disclosure will be described in detail with reference to embodiments thereof as illustrated in the accompanying drawings and figures, in which like reference numerals are used to indicate identical or functionally similar elements. References to “one embodiment”, “an embodiment”, “an example embodiment”, etc., indicate that the embodiment described may include a particular feature, structure, or characteristic, that every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following examples are illustrative, but not limiting, of the present invention. Other suitable modifications and adaptations of the variety of conditions and parameters normally encountered in the field, and which would be apparent to those skilled in the art, are within the spirit and scope of the invention.

It will be understood that like or analogous elements and/or components, referred to herein, may be identified throughout the drawings with like reference characters.

In accordance with the present disclosure, the basic operation of a vaporizer device has two modes wherein a user can select with a rocker power switch for two distinct modes of operation, namely (1) Oil Mode and (2) Leaf Mode. An additional embodiment enables an advanced mode that allows for a wide range of temperature selection so that a user may more fine-tune and select a temperature for vaporization. Oil Mode has pre-set power settings that are optimized for the vaporization of oil, and Leaf Mode has pre-set power settings that are optimized for the vaporization of leaf. A user can adjust the power settings for these two modes using the Plus and Minus buttons and can then start heating by pressing the Go button. The present invention will heat to the preset power settings configured from the user's input and will then deliver the appropriate power to the induction coil, which heats the induction cups through induction.

The induction circuitry will maintain the power to a nail at +/−1% of the programmed power adjusting the power delivered to the induction cup. This power accuracy also relates to temperature accuracy during a vaporization. Temperatures are not used though with calibrations or user settings, since the average temperature of the induction cup will vary based off the environmental temperature, and the amount of material that is desired to be vaporized.

The induction circuitry will also calculate the cooling of the induction cup, to facilitate consecutive heating sessions initiated by a user, the induction coil will heat the induction cup to the appropriate power compensating for the previously heated induction cup.

The present device has an advanced mode, that includes more control over the power settings, and heating time. This mode is activated by engaging the buttons in a certain combination during powering on/off.

The present device will also contain multiple power calibration curves that are dependent on the induction cups used, either the ceramic or crystal. This embodiment includes a ceramic induction cup composed of a titanium cup that is coated with a ceramic layer to provide an inert vaporization surface. The crystal induction cups are composed of a quartz induction cup with a metal ring bonded to it to be heated by the induction circuitry. The selected induction cup power calibration curves can be selected by the user holding down the user interface buttons in a certain combination while the unit is on.

Induction heating is the process of using a magnetic field to apply thermal power to an electrically conductive material. Induction uses a magnetic oscillating field to create an alternating magnetic flux in an electrically conductive material located within the field. This oscillating magnetic flux in the electrically conductive material will generate an internal electric field.

This electrical field will create an internal current, commonly referred to as Eddy Currents, where the current oscillates with the same frequency as the oscillating magnetic field. These internal Eddy Currents will create heat due to the internal resistance of the electrically conductive material, the same method that resistive heating works in most traditional heating elements.

In practical applications, induction heating is carried out with a metallic conductor placed in an alternating magnetic field provided by an AC powered electric coil. The heating power, heat rate, and efficiency will be dependent on: the coil design, the electrical properties of the inductive heating material, the position of the inductive heating material relative to the coil, the applied AC current frequency, and the applied AC current amplitude.

The present invention contains an induction assembly composed of an induction coil that is wired to the induction circuitry. The induction circuitry is housed in the body of the device, with the induction coil protruded into the cavity in the glass top. The glass top along with multiple silicone seals isolate the electronics from any produced vapor, or outside contaminants.

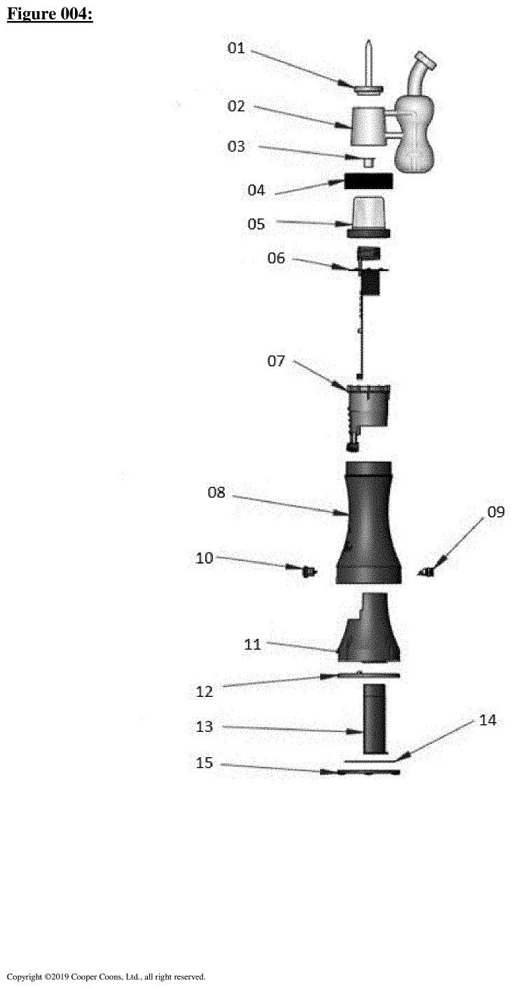

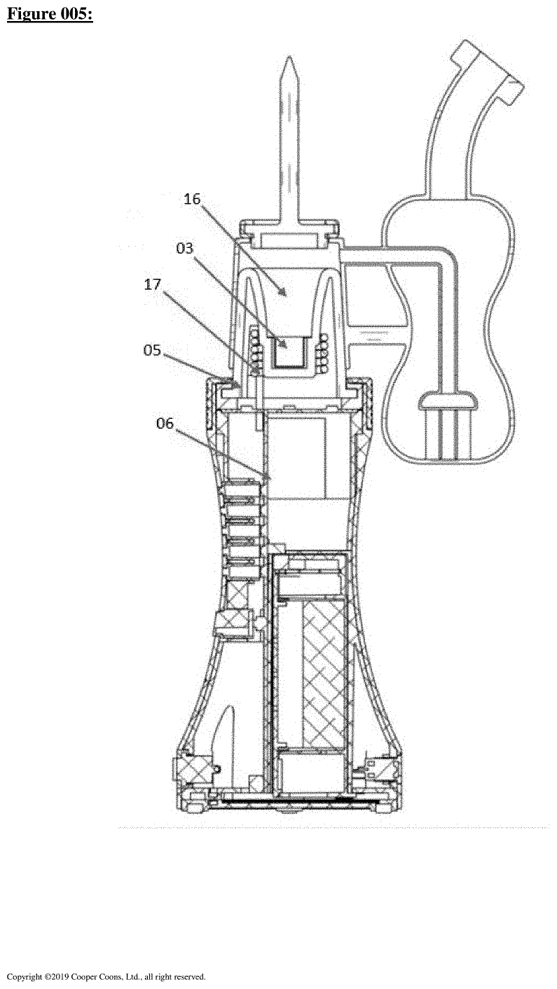

FIG. 4 depicts an explosion view of the connected elements of the present invention that includes in a sequential order of connections a carb tool 01, percolator 02, induction cup 03, locking collar 04, glass top 05, induction circuitry 06, internal chassis-A 07, body shell 08, charging connector 09, power switch 10, internal chassis-B 11, cover plate 12, battery pack 13, reference card 14, and a base plate 15.

The glass top also has another cavity that houses the induction cup 03. The glass top 05 ensures the induction cup 03 is positioned in an optimal position relative to the induction coil 17. This cavity which houses the induction cup is referred to as the vaporization chamber 16, since it is where vapor is produced from the induction cup 03.

The induction is then carried out by the induction circuitry generating an AC electric current in the induction coil, which then creates an alternating magnetic field in the vaporization chamber which heats the induction cup through induction heating.

Regarding the present invention, related to the induction cup design, it is unique among induction vaporizers because of the design of a vaporization chamber, and the material to be heated. The present invention utilizes an induction cup, which is a flanged cup that has either a partially or full metallic exterior, and an interior composed of an inert material. The outer metallic exterior will be heated by induction and transfer the heat to the interior inert material. This inert material is either a ceramic-based or glass-based material.

The use of an inert material for the interior of the induction cup is necessary to ensure the heated oil or leaf will not interact with the induction cup materials. The use of inert material is commonly known in our industry to preserve the flavor of the vapor produced and is easier to clean and maintain than a metal surface.

The present invention has been designed to work with two specific types of induction cups; the Ceramic Induction Cup, and the Crystal Induction Cup. Other alternative cup designs, including a pure metal induction cup, are also possible, but these designs have shown to be the best in terms of manufacturing, and functionality.

Regarding the ceramic induction cup 03, the present element is composed of a pure titanium cup that is vapor deposited with an alumina ceramic coating. The titanium cup will be heated by the induction heating process and transfers the heat to the ceramic coating.

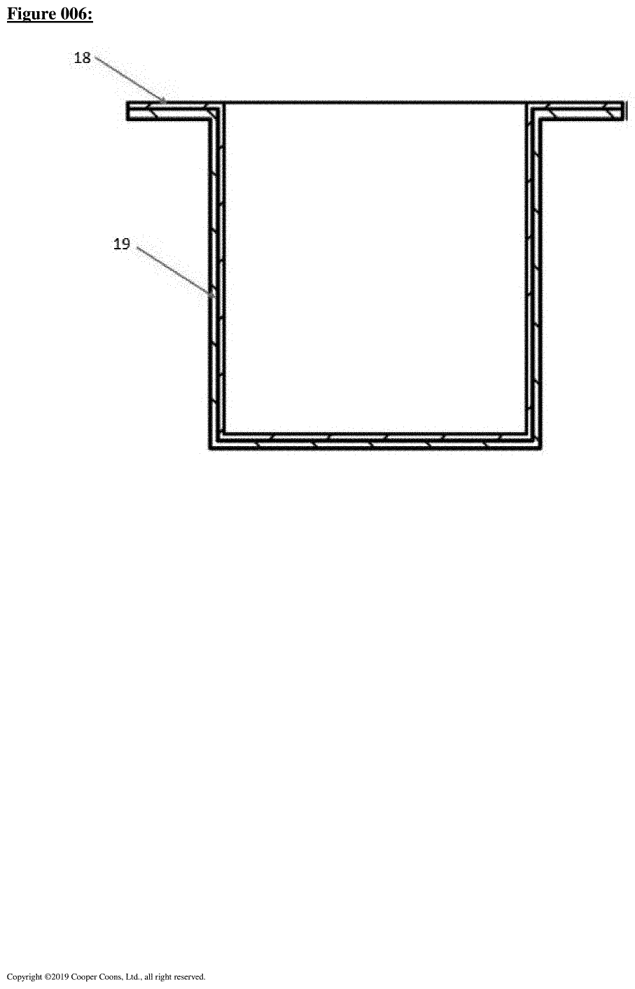

Regarding the crystal induction cup, the present element is composed of a quartz glass cup that has a stainless-steel ring pressed onto it when the quartz glass is still hot and malleable. The stainless-steel is heated by the induction heating process and transfers the heat the quartz glass cup. Regarding FIG. 6 depicts a ceramic coating 18 that surrounds and contains a metal cup 19 for heat transition to the product.

Vaporizers typically are described as heated by either convection or conduction. Convection vaporizers use heated air that passes through the desired vaporized material to extract the vapor from the material, while conduction heats the material directly from a heated surface to extract the vapor.

In practice, many conductive vaporizers have a noticeable amount of airflow in the heating chamber, which mixes fresh air with the desired vaporized material. The airflow in conductive vaporizers is also a by-product of the device's design, where the vaporizer design has un-perfect seals which causes the airflow. In summary, most vaporizers are either purposefully or accidently designed to be a combination of both conduction and convection.

The present invention and method thereof allow for a design that keeps the vaporization chamber completely sealed, and outside air does not directly pass through the induction cup. As such, the present device creates anaerobic vaporization, where the induction cup has a low amount of oxygen present during vaporization.

The anaerobic vaporization of the device causes the induction cup to be heated quickly by induction, causing the rapid temperature which heats the air and reduces the local vapor pressure. At the same time, the material in the induction cup will begin to produce vapor, replacing the air that is heated in the induction cup.

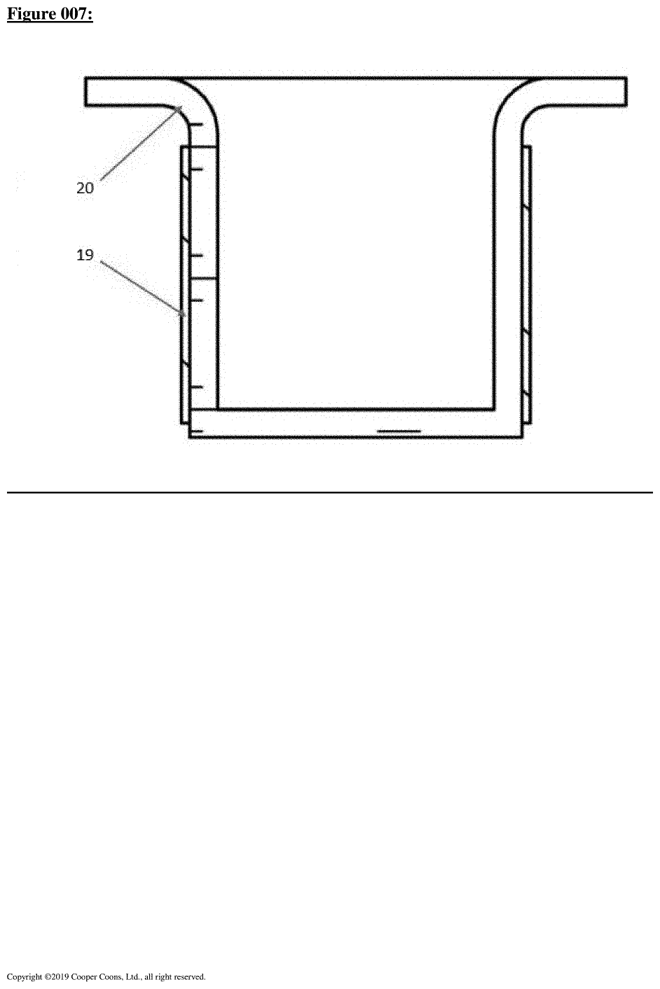

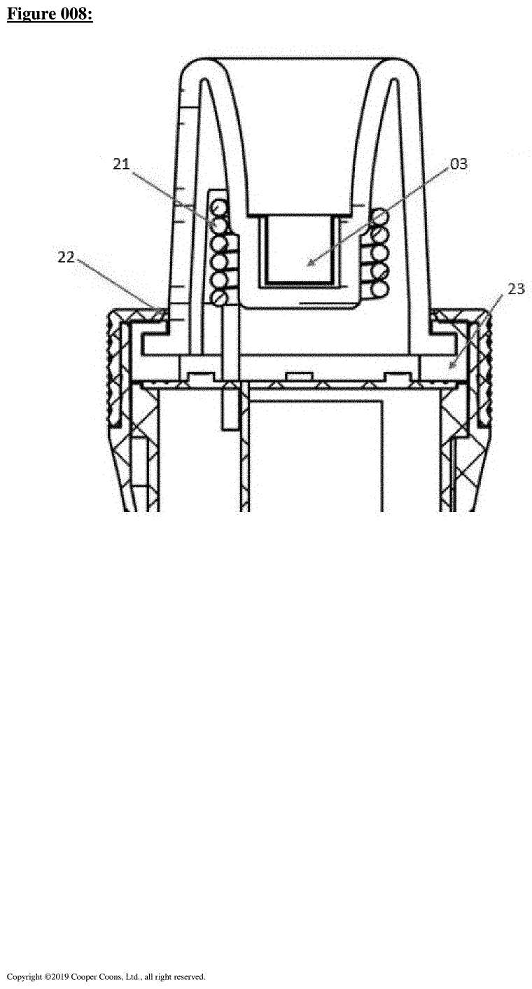

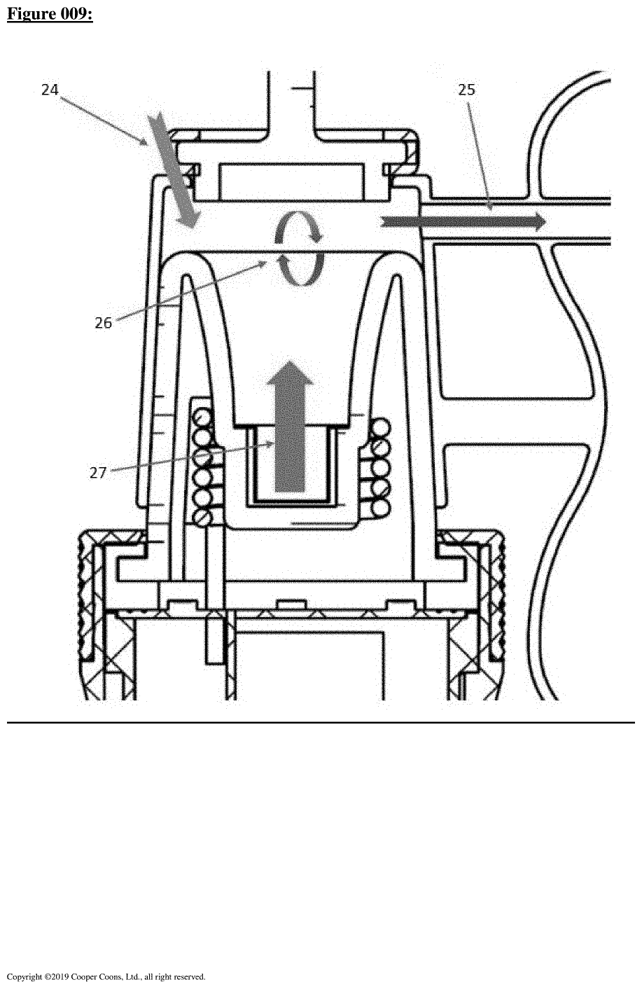

This described method produces vapor will then mix with air above the induction cup, allowing the vapor enough time to cool to a temperature where oxidation is less probable. The vapor that is extracted from the induction cup will be replaced by more vapor produced from the material until the material is exhausted of vapor. FIG. 7 depicts a quartz cup 20 articulated with metal tube 19 for construction of the present apparatus.

This anaerobic vaporization results in a less chances of combustion or oxidation of vaporized products. This anaerobic vaporization preserves both the flavor and purity of the produced vapor due to the induction coil 21 heating product within an induction cup 03 and encapsulated by an adhesive seal 22 and silicone seal 23. This anaerobic vaporization will also result in a higher vaporization efficiency, in the amount of vapor that is produced from a given amount of material due to the lack of oxygen reducing the local vapor pressure of the area inside the induction cup, thereby increasing the amount of vapor that can be extracted from the material.

FIG. 9 relates to a depiction of anaerobic vaporization of material and product within the present invention with outside airflow 25, with a mixing zone of air and vapor 26, from produced valor 27 from the induction cup 03, followed by an exhaust of vapor and air 25 for the user to inhale.

Temperature measurement systems are commonly systems that use a temperature measurement device to take readings of the system either periodically or continuously. Predictive systems are not as common and use non-direct methods to “predict” the temperature of the system.

Direct temperature measurements have many advantages since they can take accurate temperature measurements directly but may not be useable in specific scenarios. The present invention does not use a direct temperature measurement system due to the need to isolate electronics from the vaporization chamber, and the errors that may result from the strong alternating magnetic field in the vaporization chamber.

However, the present invention utilizes predictive temperature measurement system that uses the electric heating power, along with heat transfer calculations to predict the heating of the induction cups. Each induction cup type must be calibrated to account for a variable heating rate, the heat transfer inside the induction cup, and the cooling rate of the induction cup. Each of these values are used to predict the heating and cooling of the induction cup so that after consecutive heating sessions, the induction cup is accurately heated to the selected power setting.

Traditional vaporizers are only heated using a constant wattage for a controlled amount of time; this results in consecutive heating sessions result in hotter vapor at the later heating sessions.

This predictive temperature measurement allows the present invention to be accurate and give a repeatable experience during each vaporization.

The foregoing description merely explains and illustrates the invention and the invention is not limited thereto except insofar as the appended claims are so limited, as those skilled in the art who have the disclosure before them will be able to make modifications without departing from the scope of the invention.

We claim:

1. An induction powered vaporizer, comprising:
a. capable of vaporizing both liquid and solid products;
b. liquid or solid products can either be preloaded into an induction cup, or loaded into device and induction cup pre-heated by device;
c. utilizes circuitry to provide induction heating to an inserted cup, which directly heats the desired solid leaf or liquid oil material;
d. induction heating of induction cup without any electrical connections to material directly;
e. novel air-flow design, and circuitry control of the induction vaporizer;
f. induction cup housed in a glass housing creating a unique vaporization chamber to protect the electronics of device from vapor byproducts;
g. glass housing of induction cup in a well, to produce a low-oxygen environment when heating occurs;
h. glass or ceramic induction cup of bonded metal ring wherein no product contacts heating elements directly to enable the inert material to maintain taste and purity of vapor;
i. metal induction cups coated or bonded with a ceramic;
j. non-metallic induction cup bonded to a metal ring, wherein the non-metallic cup is the surface directly heating the loaded material;
k. non-metallic cup inserted into a metal induction cup used as the vaporization cup, wherein a non-metallic cup is the surface directly heating the loaded material, but non-metallic cup being heated by the metal induction cup directly by heating element of device;
l. heating profile and elements controlled by user via control buttons indicated by LED display;
m. replaceable battery pack, or powered directly from wall-outlet or alternative power source;
n. interactive user LED display, and multiple user activated buttons or switches;

2. A vaporization device, comprising: a) a vapor conduit that carries vaporized solid or liquid material components to an operator who inhales; b) a material chamber in which components of a solid leaf or liquid oil material are vaporized; and c) a temperature controlling intake having an ambient air intake port; d) wherein said temperature controlling intake is in fluid communication with said solid or liquid material chamber and said solid or liquid material chamber is in fluid communication with said vapor conduit; and e) wherein said temperature controlling intake comprises a temperature controller that changes its displacement with temperature; and further comprising: (f) a heat chamber in heat transfer contact but not fluid contact with said temperature controlling intake, (g) induction heating of induction cup without any electrical connections to material directly; (h) induction cup housed in a glass housing creating a unique vaporization chamber to protect the electronics of device from vapor byproducts; (i) glass housing of induction cup in a well, to produce a low-oxygen environment when heating occurs; (j) glass or ceramic induction cup of bonded metal ring wherein no product contacts heating elements directly to enable the inert material to maintain taste and purity of vapor; (k) metal induction cups coated or bonded with a ceramic; (l) non-metallic induction cup bonded to a metal ring, wherein the non-metallic cup is the surface directly heating the loaded material; (m) non-metallic cup inserted into a metal induction cup used as the vaporization cup, wherein a non-metallic cup is the surface directly heating the loaded material, but non-metallic cup being heated by the metal induction cup directly by heating element of device; (n) heating profile and elements controlled by user via control buttons indicated by LED display; (o) replaceable battery pack, or powered directly from wall-outlet or alternative power source; (p) interactive user LED display, and multiple user activated buttons or switches.
